(19) United States
(12) Patent Application Publication
(10) Pub. No.: US 2021/0215724 A1
(43) Pub. Date: Jul. 15, 2021

(54) METHOD OF TREATING PLASMA FOR USE

(71) Applicant: Oklahoma Blood Institute, Oklahoma City, OK (US)

(72) Inventor: Charles Mooney, Oklahoma City, OK (US)

(21) Appl. No.: 17/143,699

(22) Filed: Jan. 7, 2021

Related U.S. Application Data
(60) Provisional application No. 62/959,371, filed on Jan. 10, 2020.

(51) Int. Cl. G01N 33/80; G01N 33/537; G01N 33/543; G01N 33/68

(52) U.S. Cl. CPC G01N 33/80; G01N 33/6854; G01N 33/54333; G01N 33/537

(57) ABSTRACT

A method treats human plasma obtained from any donor and renders the plasma, or serum derived therefrom, compatible with any recipient regardless of the recipient's blood group. Broadly, the method mixes type-AB red blood cells with blood plasma containing antibodies which are anti-A antibodies, anti-B antibodies or both so as to form a blood-plasma mixture, which can be separated to obtain a refined plasma which is lower in antibodies than the initial blood plasma.

### CROSS REFERENCE TO RELATED APPLICATION

This application claims the benefit of U.S. Provisional Patent Application No. 62/959,371 filed on Jan. 10, 2020, which is hereby incorporated by reference.

### FIELD

The present invention is a method for treating human blood plasma prior to further processing the plasma into serum and, more particularly, is a method for removing blood group antibodies from the plasma to avoid incompatibility between donors and recipients of different blood groups.

### BACKGROUND

Blood is a heterogeneous system in which a variety of cells are suspended in an aqueous solution (plasma) composed of proteins, lipoproteins and lipids in colloidal suspension and organic metabolites in solution. The cells include red cells (erythrocytes), white cells (leukocytes) and platelets (thrombocytes). Plasma is the solution or liquid which remains after the cells have been removed from the blood. Present in the plasma are various proteins, including antibodies secreted by the white cells as an immunological response to the presence of antigens, as well as proteins responsible for other essential physiological processes, such as the clotting factors required for hemostasis.

The major human blood group or type is determined by the presence of ABH antigens on the cell surface of the red blood cells. The antigens are oligosaccharides attached to glycolipids or glycoproteins in the plasma membranes of the red blood cells. The antigenic structure is of three types, referred to as “A”, “B” and neither “A” nor “B” (that is “H” antigen or type “0” blood). Either the A antigen, the B antigen, or both the A and B antigen may be present on the surface of an individual's red blood cells. Every individual's blood will contain naturally occurring antibody (primarily IgM) against the AB antigens which are not present on his red blood cells. The situation is summarized in Table 1, which follows.

TABLE 1

| Blood Group | Antigen on Red Cell | Antibodies in Blood |
|---|---|---|
| A | A antigen substance | anti-B antibodies |
| B | B antigen substance | anti-A antibodies |
| AB | A and B antigen substance | Neither anti-A nor anti-B |
| | | antibodies |
| O | Neither A nor B antigen | Both anti-A and anti-B |
| | substance | antibodies |

Serum used in tissue culture products that will later be used in humans should not contain anti-A or anti-B antibodies for similar incompatibility reasons. When plasma is used in a cell culture, incompatibility between the plasma donor and the recipient of the final cell culture product arises whenever the plasma donor's blood includes antibodies to the AB antigens. For example, plasma from a donor having type-A blood cannot be infused into a recipient having group B blood since the donor's plasma includes anti-B antibodies which would react with the B antigen on the recipient's red blood cells. Thus, plasma from a donor having type-O blood, which includes both anti-A and anti-B antibodies is incompatible with a recipient having any blood type A or blood type B. Conversely, plasma from a donor having type-AB blood, which includes neither anti-A nor anti-B antibodies, is compatible with any recipient, regardless of blood group. Because of the potential for incompatibility, it has heretofore been necessary to only use type AB plasma when performing cell cultures for products that will later be injected into patients with various blood types.

While plasma and serum are sometimes informally used interchangeably, they are distinct from each other. Serum and plasma both come from the liquid portion of the blood that remains once the cells are removed, but that is where the similarities end. Plasma is the liquid that remains when clotting is prevented with the addition of an anticoagulant. Serum is the solution that remains after the blood has been clotted and the clot separated from the liquid.

Historically, blood plasma or serums have had to match blood types of the recipients or alternatively, human AB type blood could be used to produce plasma or serum compatible with all recipients. This AB blood type can be used because plasma from these donors do not contain the naturally occurring anti-A and anti-B antibodies. This attribute makes them the universal donor for plasma and serum. Their plasma can be given to patients of every other blood type. However, only 4% of the population has this AB blood type. The low frequency of the blood type combined with using these donors for transfusion and tissue culture media, has led to a global shortage of AB plasma and serum.

For the above reasons, it would be desirable to provide a treatment for removing the antibodies from plasma from all other blood types to provide a “universal” plasma which could be used for cell therapy cultures regardless of recipients' blood type. Such treatment, however, must be highly specific to ensure that essential blood proteins are not removed or inactivated. For example, albumin that is in human plasma must maintain its viable chemical structure to provide the cultured cells with the nutrients needed to expand the population of cells to a therapeutic dose. Additionally, it is imperative that other contaminates not be introduced to the plasma causing product contamination or failure.

### SUMMARY

The present disclosure relates to removing isohemagglutinins from human plasma/serum prior to using the plasma/serum as a supplement in human cell cultures. This invention allows the plasma/serum to be used without worry of incompatibility with human blood types.

The present disclosure provides a method of treating human plasma obtained from any donor which renders the plasma or product compatible with being injected into any recipient regardless of the recipient's blood group. The method comprises a step of mixing at least one unit of type-AB red blood cells with at least one unit of blood plasma containing antibodies which are anti-A antibodies, anti-B antibodies or both so as to form a blood-plasma mixture containing sufficient antigen to bind with any corresponding antibody. For example, a blood-bag container can be attached by sterile welding tubing, which is integrally attached to the blood bag container of each blood product so as to connect them together in a functionally closed system allowing the contents of packed red blood cell units to be sterilely added to the plasma unit. The volume of packed red blood cells added to the plasma unit must be of sufficient quantity of type-AB, packed red blood cells in relation to the unit of blood plasma so that there are sufficient antigens to bind with any corresponding antibody. In embodiments, the unit(s) of blood plasma will generally be greater than 200 ml and the unit(s) of type-AB red blood cells will be sufficient to supply antigen to bind with any corresponding antibodies in the unit(s) of blood plasma. The type-AB red blood cells and plasma typically will be approved or licensed by the appropriate authority. For example, in the U.S. they will be approved or licensed by the U.S. Food and Drug Administration. The resulting blood-plasma mixture is periodically mixed to ensure that the antigen comes into contact with the corresponding antibody over a period of more than about 5 hours at a temperature of from about 0° C. to about 10° C. so that the fragment antigen-binding (FAB) region of the cold reacting immunoglobulin M (IgM) antibodies are bound to antigens on the surface of the type-AB red blood cells in the AB packed red blood cell unit.

After sufficient mixing has occurred, the blood plasma is separated from the blood-plasma mixture to obtain a refined plasma which is lower in antibodies than the blood plasma. The refined plasma will generally have the anti-A and/or anti-B antibodies reduced to a level that is not detectable using serological detection techniques. The use of blood products combined through a functionally closed system results in a refined plasma suitable for injection into humans.

In most applications, the refined plasma is substantially free of antibodies to at least below a level detectable by the standard reverse blood group typing serological methods. In the method, there will be generally at least two units of blood plasma for each unit of type-AB red blood cells, and more typically 3 or even 4 units of blood plasma for each unit of type-AB packed red blood cells. Generally, the type-AB red blood cells will be introduced as a unit of type-AB packed red blood cells. Further, the blood plasma can be plasma obtained from type-A blood, plasma obtained from type-B blood, or plasma obtained from type-O blood. In embodiments, the blood plasma is obtained from type-O blood.

In embodiments, the periodic mixing more typically will be over a period of more than about 8 hours. In embodiments, the temperature more typically will be from about 2° C. to about 8° C.

In the method, the separation can be carried out by a refrigerated centrifuge at a temperature of from 0° C. to 10° C., and preferably from 2° C. to 8° C.

In embodiments where an appropriate anticoagulant has been added in the blood plasma, the refined plasma from the above method can be converted to serum by mixing the refined plasma with injectable grade calcium carbonate and injectable grade thrombin. Such mixing results in activation of clotting factors resulting in a fibrinogen clot. The clotted fibrinogen is then separated out to leave a serum, by sterile welding an empty sterile blood bag keeping a functionally closed sterile product which is suitable for injection into humans without regard to blood type of a recipient.

### BRIEF DESCRIPTION OF THE DRAWINGS

FIG. 1 is a schematic diagram of a combination of plasma from type-O donors with red blood cells from type-AB donors in accordance with embodiments of the invention as described in the Example.

### DETAILED DESCRIPTION

The present disclosure may be understood more readily by reference to these detailed descriptions. Numerous specific details are set forth in order to provide a thorough understanding of the various embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawing is not necessarily to scale and the proportions of certain parts have been exaggerated to better illustrate details and features of the present disclosure.

Before discussing the presently disclosed inventive concepts in detail by way of exemplary description, drawings and example, it is to be understood that the inventive concepts disclosed herein are not limited in application to the details of construction and the arrangement of the compositions, formulations, steps, or components set forth in the following description or illustrated in the drawings and/or examples. The presently disclosed inventive concepts are capable of other embodiments or of being practiced or carried out in various ways. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting except where indicated as such.

All of the compositions, devices, systems, and/or methods disclosed herein can be made and executed without undue experimentation in light of the present disclosure. Although certain steps are described herein and illustrated in the FIGURES as occurring sequentially, some steps may occur simultaneously with each other or in an order that is not depicted.

As utilized in accordance with the present disclosure, the following terms, unless otherwise indicated, shall be understood to have the following meanings: the use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements, or method steps.

Throughout this disclosure, the terms “about”, “approximate”, and variations thereof, are used to indicate that a value includes the inherent variation or error for the device, system, the method being employed to determine the value, or the variation that exists among the study subjects.

The present disclosure provides a method resulting in a refined human blood plasma, and/or blood serum obtained from the refined plasma, compatible with any recipient regardless of the recipient's blood group.

Similarly, serum used in tissue cultured products that will later be used in humans should not contain anti-A or anti-B antibodies. Serious reactions have been known to occur where such products were developed using serum containing anti-A or anti-B antibodies.

Donors with blood type-AB do not have either anti-A or anti-B antibodies whereas, donors with blood type 0 have both antibodies. Red blood cells from blood type-AB donors have both A and B antigen substance attached to their cell walls, while blood type-O red blood cells have neither antigen. When plasma is used in a cell culture, incompatibility between the plasma donor and the recipient of the final cell culture product arises whenever the plasma donor's blood includes antibodies to the AB antigens. For example, plasma from a donor having type-A blood cannot be infused into a recipient having type-B blood since the donor's plasma includes anti-B antibodies which would react with the B antigen on the recipient's red blood cells. Thus, plasma from a donor having type-O blood, which includes both anti-A and anti-B antibodies is incompatible with a recipient having any blood type-A or blood type-B.

Conversely, plasma from a donor having type-AB blood, which includes neither anti-A nor anti-B antibodies, is compatible with any recipient, regardless of blood group. Because of the potential for incompatibility, it has heretofore been necessary to only use type-AB plasma when performing cell cultures for products that will later be injected into patients with various blood types.

The current disclosure takes advantage of the discovery that unwanted antibodies in plasma units can be removed from a solution by adding the antigen for a packed red blood cell to which the antibody has been programmed to attach to the antigen. By mixing packed red blood cells from blood type-AB donors into a pool of plasma from blood type-O donors, blood type-A donors or blood type-B donors, the anti-A and/or anti-B antibodies from the plasma will attach to the A and B antigen on the surface of the red blood cell. If enough antigen is present all of the antibodies in the solution will be bound to the surface of the red blood cell. The red blood cell covered with bound antibody can be removed from the solution to provide an antibody free solution. Accordingly, the present disclosure provides a method of treating human plasma obtained from any donor which renders the plasma or product compatible with any recipient regardless of the recipient's blood group. More specifically, the method comprises mixing at least one unit of type-AB red blood cells with at least one unit of blood plasma containing antibodies, which are anti-A antibodies, anti-B antibodies or both, so as to form a blood-plasma mixture. The unit of type-AB red blood cells will often be a unit of type-AB whole blood, thus containing type-AB antigens on the surface of red blood cells. As used in herein, “unit” is a generic term for a volume of the substance, and a unit of one substance is to be interpreted as being the same unit and volume when compared to or in association with a unit of another substance. In other words, one unit of type-AB whole blood mixed one unit of blood plasma, refers to both the whole blood and blood plasma being present in the same volume amount, for example 500 ml whole blood and 500 ml of blood plasma. Further, 2 units of whole blood mixed with 1 unit of blood plasma, means that twice the volume of whole blood is used as blood plasma.

Typically, the method will utilize type-O blood plasma; however, it is within the scope of the method to use type-A blood plasma or type-B blood plasma. Furthermore, the method typically used type-AB red blood cells, however, it is within the scope of the method to use type-A red blood cells or type-B red blood cells, as long as there is incompatibility between the red blood cells and the blood plasma. The blood plasma used is plasma that has been separated out of whole blood of the associated blood type and thus contains the antibodies that are anti-A antibodies, anti-B antibodies or both, depending on the blood type. Additionally, the plasma will contain various soluble proteins, such a fibrinogen. Accordingly, the blood plasma will include an anticoagulant, which is the same as the anticoagulant in the type-AB red blood cells.

While the ratio of the volume of whole blood used to supply type-AB red blood cells to the volume plasma can be 1:1, it has been found that the method can be successful for ratios of 1:2, 1:3, 1:4 units or since the number of AB antigens on a red blood cell is different for each individual, other ratios of type-AB red blood cells can be used as appropriate. The ratio of antigen must simply be greater than the ratio of antibody to ensure adequate antibody removal.

The introduction of type-AB red blood cells to the blood plasma can be at room temperature or can be at a refrigerated temperature of from about 0° C. to about 10° C., and more typically, from about 2° C. to about 8° C. If the introduction is at room temperature, the resulting blood-plasma mixture should be subsequently reduced in temperature to the refrigerated temperature and should typically remain at the refrigerated temperature for an interaction period of at least 4 hours, typically at least 5 hours, or at least 6 hours, and more typically at least 8 hours or about 8 hours.

Periodically during the interaction period, the blood-plasma mixture should be further mixed to ensure a good interaction of the antibodies to the antigens on the surface of the red blood cells so that substantially all the antibodies are bound to the antigens. As will be appreciated, the ratio of type-AB red blood cells to blood plasma in the blood-plasma mixture should be sufficient to bind all or substantially all the anti-A and anti-B antibodies present in the blood plasma.

Once the interaction period is completed, the blood plasma is separated from the blood-plasma mixture to obtain a refined plasma which is lower in antibodies than the blood plasma. For example, the separation can be carried out by a refrigerated centrifuge at a temperature of from 0° C. to 10° C., and preferably from 2° C. to 8° C.

As will be realized, the refined plasma can not only include the blood plasma introduced at the start of the method, now substantially free of anti-A and anti-B antibodies, but also additional plasma from any type-AB whole blood used to provide the type-AB red blood cells. In most applications, the refined plasma is substantially free of antibodies to at least below a level detectable by the standard serological reverse blood grouping methods; thus, as used herein “substantially free of antibodies” means that antibodies are only present at a level below that detectable by standard serological reverse blood grouping methods. Such test can include, enzyme-linked immunosorbent assay (ELISA), agglutination, precipitation, complement-fixation, fluorescent antibodies and chemiluminescence methods, as are known in the art.

As indicated above, the method can be successful for ratios of 1:2, 1:3, 1:4 or other units of whole type-AB whole blood to blood plasma. Thus, whereas the type-AB whole blood might produce at most one unit of a “universal” plasma, the use of the whole blood in the current process can produce over 4 units, and even up to 5 units, of “universal” plasma based on the 4 units of plasma introduced into the method and the plasma obtained from the 1 unit of type-AB whole blood.

After such treatment, the refined plasma can be stored or used in the conventional manner. In the case where blood serum is desired, the refined plasma can be further treated. In embodiments where an appropriate anticoagulant has been added in the blood plasma and/or whole blood, the refined plasma from the above method can be converted to serum by mixing the refined plasma with calcium carbonate and thrombin. Such mixing results in clotting of the fibrinogen. The clotted fibrinogen is then separated out to leave a serum, which is suitable for use without regard to blood type of a recipient.

The following prophetic example provides further information and details on how the method of this disclosure may be carried out to produce a universal plasma and/or universal serum. The prophetic example is offered by way of illustration of the process and is not meant to be limiting; rather, the scope is defined by the claims.

### Example

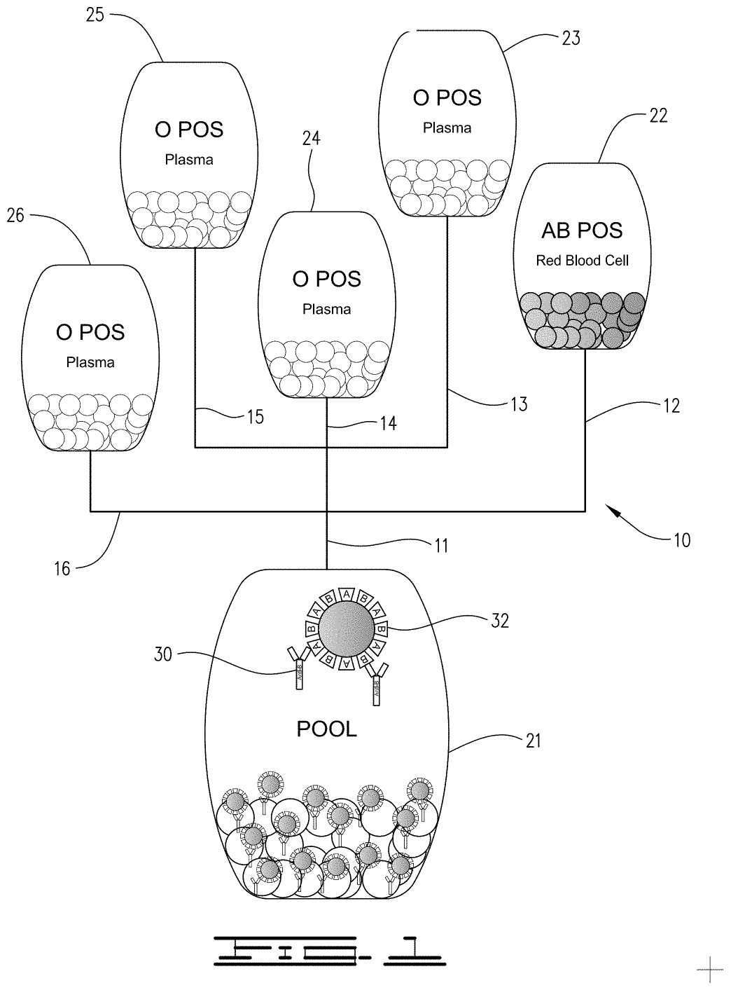

Referring to FIG. 1, a commercially available multi-lead fluid transfer tubing set 10 having leads 11, 12, 13, 14, 15 and 16 is provided. An empty large volume transfer bag 21 is sterile welded to lead 11 of tubing set 10. A bag 22 of blood type-AB whole blood is sterile weld to lead 12 of tubing set 10. To the remaining leads 13, 14, 15 and 16, one plasma unit (bags 23, 24, 25 and 26) is sterile weld to each lead. Each plasma unit 23, 24, 25 and 26 contains plasma obtained from type-O blood. The connections are made with the pathways of the leads closed.

Once the connections have been made, the pathways are open so that there is fluid flow from bags 22, 23, 24, 25 and 26 into transfer plasma bag 21. For example, empty transfer bag 21 can be placed onto a level surface, and bags 22-26 can be raised so that the red blood cells (RBC) from bag 22 and plasma from bags/units 23-26 are above the empty bag thus allowing gravity flow to force the contents of each of the plasma units and the RBC unit into transfer bag 21.

Once the RBC and plasma have been transferred into transfer bag 21, transfer tubing set 10 can be removed from transfer bag 21. Transfer bag 21 that now contains substantially 1-unit of type-AB red blood cells and 4-units of type-O plasma. Using a swirling motion mix the contents. Place transfer bag 21 containing the mixed solution into a 2-6° C. environment. The mixture remains in the 2-6° C. environment typically for a minimum of 4-8 hours of incubation. During the incubation, the contents are mixed periodically by gently swirling the contents. During the incubation, anti-A and anti-B antibodies 30 from the type 0 plasma will attach to the A and B antigens 32 on the surface of the red blood cells as shown in FIG. 1.

After typically 4-8 hours of cold environment incubation, sterile weld an empty transfer bag onto transfer bag 21 containing the mixture. Place the mixture into a refrigerated centrifuge and centrifuging until there is a clear interface between the red blood cells and the plasma. Remove the unit from the centrifuge and open the fluid pathway between the empty transfer bag and transfer bag 21. Using pressure, push the plasma into the empty transfer bag. The plasma in the transfer bag is now antibody reduced and can be used as plasma or converted to serum by adding calcium carbonate and thrombin.

Other embodiments of the present invention will be apparent to one skilled in the art. As such, the foregoing description merely enables and describes the general uses and methods of the present invention. Accordingly, the following claims define the true scope of the present invention.

What is claimed is:

1. A method of treating human plasma so as to render the plasma compatible with being injected into any recipient regardless of blood group, the method comprising:
mixing at least one unit of type-AB red blood cells with at least one unit of blood plasma to form a blood-plasma mixture, wherein the at least one unit of blood plasma contains antibodies selected from the group of anti-A antibodies, anti-B antibodies and combinations;
continuing the mixing the blood-plasma mixture at least intermittently at a temperature of from about 0° C. to about 10° C. such that antigen in the type-AB red blood cells contact the antibodies over a mixing period of more than about 4 hours so that the antibodies are bound to the antigens on the surface of the type-AB red blood cells;
after the mixing period, separating blood plasma from the blood-plasma mixture to obtain a refined plasma which is lower in antibodies than the blood plasma.

2. The method of claim 1, wherein the refined plasma is substantially free of antibodies.

3. The method of claim 1, wherein there are at least two units of blood plasma for each unit of type-AB red blood cells.

4. The method of claim 1, wherein there are at least three units of blood plasma for each unit of type-AB red blood cells.

5. The method of claim 1, wherein the type-AB red blood cells are type-AB packed red blood cells.

6. The method of claim 1, wherein the blood plasma is selected from the group of blood plasma obtained from type-A blood, blood plasma obtained from type-B blood, and plasma obtained from type-O blood.

7. The method of claim 1, further comprising:
adding an anticoagulant to the blood plasma,
converting the refined plasma to serum by mixing the refined plasma with injectable grade calcium carbonate and injectable grade thrombin to produce a fibrinogen clot; and
separating out the fibrinogen clot to leave a serum.

8. The method of claim 1, wherein the blood plasma is obtained from type-O blood.

9. The method of claim 1, wherein the mixing period is at least 8 hours and is at a temperature of from about 2° C. to about 8° C.

10. The method of claim 9, wherein the separation can be carried out by a refrigerated centrifuge at a temperature of from 0° C. to 10° C., and preferably from 2° C. to 8° C.

11. The method of claim 10, wherein:
the type-AB red blood cells are type-AB packed red blood cells;
the blood plasma is obtained from type-O blood; and
there are at least three units of blood plasma for each unit of type-AB red blood cells.

12. The method of claim 11, wherein there are at least four units of blood plasma for each unit of type-AB red blood cells.

13. The method of claim 12, wherein the refined plasma is substantially free of antibodies.

14. The method of claim 13, further comprising:
adding an anticoagulant to the blood plasma,
converting the refined plasma to serum by mixing the refined plasma with injectable grade calcium carbonate and injectable grade thrombin to produce a fibrinogen clot; and
separating out the fibrinogen clot to leave a serum.
